(19) United States
(12) Patent Application Publication
(10) Pub. No.: US 2014/0134747 A1
(43) Pub. Date: May 15, 2014

(54) METHODS FOR DETERMINING IF AN ANIMAL'S METABOLISM IS KETOGENIC

(71) Applicant: NESTEC SA, Vevey (CH)

(72) Inventors: Yuanlong Pan, Chesterfield, MO (US); Steven S. Hannah, Chesterfield, MO (US); Peichuan Sun, St. Louis, MO (US); Janet R. Jackson, Columbia, IL (US)

(73) Assignee: NESTEC SA, Vevey (CH)

(21) Appl. No.: 14/076,364

(22) Filed: Nov. 11, 2013

Related U.S. Application Data
(60) Provisional application No. 61/725,202, filed on Nov. 12, 2012.

(51) Int. Cl. G01N 33/64

(52) U.S. Cl. CPC G01N 33/64
USPC 436/129

(57) ABSTRACT

The invention provides methods for determining if an animal's metabolism has been shifted to ketogenic status by collecting a first urine sample from the animal when the animal's metabolism is not in a ketogenic status; collecting a second urine sample from the animal when the animal's metabolism is possibly in a ketogenic status; analyzing the first urine sample and the second urine sample for beta-hydroxy butyrate; and determining that the animal's metabolism has been shifted to ketogenic status if the concentration of beta-hydroxy butyrate in the second urine sample exceeds the concentration of beta-hydroxy butyrate in the first urine sample by ten percent (10%) or more.

### CROSS REFERENCE TO RELATED APPLICATIONS

This application claims priority to U.S. Provisional Application No. 61/725202 filed Nov. 12, 2012, the disclosure of which is incorporated herein by this reference.

### BACKGROUND OF THE INVENTION

1. Field of the Invention

This invention relates generally to methods for determining the status of an animal's metabolism and particularly to methods for determining if an animal's metabolism is ketogenic.

2. Description of Related Art

The status of an animal's metabolism is often related to the animal's health. For example, a ketogenic diet is believed to be a valuable therapeutic approach for combating epilepsy and brain tumors. US20100310740A1 discloses ketogenic diets and methods for preparing such ketogenic diets. Similarly, ketogenic diets have been used to assist in the management of glioblastoma multiforme (GBM). Further, U.S. Pat. No. 8,124,589 discloses the use of ketogenic compounds for treatment of age-associated memory impairment. U.S. Pat. No. 7,351,736 discloses methods for producing a physiologically acceptable ketosis to treat a patient in need of therapy for one or more of Amylotrophic lateral sclerosis, Free Radical disease, Heart failure and Duchenne's muscular dystrophy. US20120252902A discloses methods for treating attention deficit hyperactivity disorder (ADHD) and related CNS disorder symptoms of impaired learning, impaired planning, impaired problem solving, impulsiveness attention deficit and aggression by administering a ketogenic material in amounts sufficient to produce a ketosis. US20080249173A1 discloses methods for treating a patient suffering from apoptosis of tissue by administering a therapeutically effective amount of one or more ketogenic compounds such that a physiological ketosis is produced sufficient to arrest said apoptosis. These therapies are only beneficial if the animal has a ketogenic metabolism. Therefore, it is important to be able to determine if an animal has a ketogenic metabolism.

Current methods for determining if an animal's metabolism is ketogenic involve using urine strips to check for ketonuria, i.e., checking for ketone bodies such as acetone in the urine. However, the concentration of ketone bodies in the urine varies depending on the animal, the animal' age, the animal's health, the environment, and the like. Merely determining the concentration of ketone bodies for an animal and comparing the concentration to known standard values is often inconclusive and can lead to a misdiagnosis that has adverse consequences on an animal's health. There is, therefore, a need for new methods for determining if an animal's metabolism is ketogenic.

### SUMMARY OF THE INVENTION

It is, therefore, an object of the present invention to provide methods for determining if an animal's metabolism is ketogenic.

It is another object of the present invention to provide methods for evaluating the affect of a comestible composition on the ketogenic status of an animal.

It is a further object of the invention to provide methods for evaluating the affect of a diet on the ketogenic status of an animal.

One or more of these and other objects are achieved using novel methods for determining if an animal's metabolism is ketogenic and/or evaluating the affect of a comestible composition or diet on the ketogenic status of an animal. These methods involve collecting a urine sample from the animal at two different times, determining the concentration of beta-hydroxy butyrate in the two urine samples, and making conclusions regarding the animal's ketogenic status and/or the affect of a comestible composition or diet on such status based upon the difference between the beta hydroxybutyrate concentrations for the two samples.

Other and further objects, features, and advantages of the invention will be readily apparent to those skilled in the art.

### DETAILED DESCRIPTION OF THE INVENTION

### Definitions

The term “animal” means a human or other animal that could benefit from a determination of the animal's ketogenic status, including bovine, canine, equine, feline, hicrine, murine, ovine, and porcine animals.

### The Invention

In one aspect, the invention provides methods for determining if an animal's metabolism is ketogenic. The methods comprise collecting a first urine sample from the animal when the animal's metabolism is not ketogenic; determining the concentration of beta-hydroxy butyrate in the first urine sample; collecting a second urine sample from the animal when the animal's metabolism is possibly ketogenic; determining the concentration of beta-hydroxy butyrate in the second urine sample; and concluding that the animal's metabolism is ketogenic if the concentration of beta-hydroxy butyrate in the second urine sample exceeds the concentration of beta-hydroxy butyrate in the first urine sample by ten percent (10%) or more.

In another aspect, the invention provides methods for evaluating the effect of a comestible composition on the ketogenic status of an animal. The methods comprise collecting a first urine sample from the animal before feeding the comestible composition to the animal; determining the concentration of beta-hydroxy butyrate in the first urine sample; feeding the comestible composition to the animal; collecting a second urine sample from the animal after feeding the comestible composition to the animal; determining the concentration of beta-hydroxy butyrate in the second urine sample; and concluding that the comestible composition caused the animal's metabolism to become ketogenic if the concentration of beta-hydroxy butyrate in the second urine sample exceeds the concentration of beta-hydroxy butyrate in the first urine sample by ten percent (10%) or more.

In another aspect, the invention provides methods for evaluating the effect of a diet on the ketogenic status of an animal. The methods comprise collecting a first urine sample from the animal before feeding the diet to the animal; determining the concentration of beta-hydroxy butyrate in the first urine sample; feeding the diet to the animal; collecting a second urine sample from the animal while feeding the diet to the animal or after feeding the diet to the animal; determining the concentration of beta-hydroxy butyrate in the second urine sample; and concluding that the diet caused the animal's metabolism to become ketogenic if the concentration of beta-hydroxy butyrate in the second urine sample exceeds the concentration of beta-hydroxy butyrate in the first urine sample by ten percent (10%) or more.

In various embodiments, the animal's metabolism is determined to be ketogenic or the comestible composition or diet has caused the animal's metabolism to become ketogenic if the amount of beta-hydroxy butyrate in the second urine sample exceeds the amount of beta-hydroxy butyrate in the first urine sample by 10%, 25%, 50%, 75%, 100%, 200%, 300%, 400%, 500%, or more. Similarly, the animal's metabolism is determined to be ketogenic or the comestible composition or diet has caused the animal's metabolism to become ketogenic if the amount of beta-hydroxy butyrate in the second urine sample exceeds the amount of beta-hydroxy butyrate in the first urine sample by from about 10 to about 300%, preferably from about 100 to about 500%, more preferably from about 300 to 2500%. Also, using other paramaters, the animal's metabolism is determined to be ketogenic or the comestible composition or diet has caused the animal's metabolism to become ketogenic if the amount of beta-hydroxy butyrate in the second urine sample exceeds the amount of beta-hydroxy butyrate in the first urine sample by 1, 2, 3, 4, 5, 6, 7, 8, 9, 10, 11, or 12 times.

The urine can be collected in any suitable manner known to skilled artisans. Generally, the urine is collected by inducing an animal to urinate into a suitable container, e.g., cups and tubes. In one embodiment, urine is collected using a catheter inserted into the animal's bladder. In other embodiments, the urine is collected using supra pubic aspiration.

The urine is analyzed for beta-hydroxy butyrate using any suitable method known to skilled artisans. Generally, a urine sample is collected and analyzed using the standard methods that determine the concentration of urine beta-hydroxybutyrate using commercially available manual or automated urine analyzers, test kits, test strips, dipsticks, and the like, e.g., test kits sold by Thermo Fisher Scientific, Noble Park, Victoria AS or by Dirui Industrial Co., Ltd, Changchun, China 130012.

### EXAMPLES

The invention can be further illustrated by the following example, although it will be understood that these examples are included merely for purposes of illustration and are not intended to limit the scope of the invention unless otherwise specifically indicated.

### Example 1

Nine (9) dogs were fed a non-ketogenic diet, for seven (7) days and urine samples were collected within six (6) hours after feeding on day seven. Then, a ketogenic diet containing medium-chain triglycerides was fed to the dogs for twenty-one (21) days and urine samples were collected within 6 hours after feeding on day twenty-one. Samples were analyzed for the presence of beta-hydroxy butyrate. Beta-hydroxy butyrate was analyzed with the Precision Xtra® Blood Glucose and Ketone Monitoring System (Abbott laboratory, Abbott Park, Illinois, USA). The results are shown in Table 1.

Referring to the results, the data shows that urine samples from dogs fed the ketogenic diet had a concentration of beta-hydroxy butyrate that was at least ten percent (10%) more than the urine samples from dogs fed a non-ketogenic diet.

TABLE 1

| | Non-ketogenic | Ketogenic |
|---|---|---|
| | Diet | Diet |
| Urine beta-hydroxy- | 27.61 | 252.94 |
| butyrate (umol/L) | | |

As used herein, ranges encompass each and every value within the range and are used to avoid having to list each and every value within the range. Any appropriate value within the range can be selected, where appropriate, as the upper value, lower value, or terminus of the range.

The invention is not limited to the particular methodology, protocols, and reagents described herein because they may vary. Further, the terminology used herein is for the purpose of describing particular embodiments only and is not intended to limit the scope of the invention.

As used herein, the singular form of a word includes the plural, and vice versa, unless the context clearly dictates otherwise. Thus, the references “a”, “an”, and “the” are generally inclusive of the plurals of the respective terms. For example, reference to “a diet” or “a method” includes a plurality of such “diets” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for determining if an animal's metabolism is ketogenic comprising:
collecting a first urine sample from the animal when the animal's metabolism is not ketogenic;
determining the concentration of beta-hydroxy butyrate in the first urine sample;
collecting a second urine sample from the animal when the animal's metabolism is possibly ketogenic;
determining the concentration of beta-hydroxy butyrate in the second urine sample; and
concluding that the animal's metabolism is ketogenic if the concentration of beta-hydroxy butyrate in the second urine sample exceeds the concentration of beta-hydroxy butyrate in the first urine sample by ten percent (10%) or more.

2. The method of claim 1 wherein the urine is collected by having the animal urinate into a container.

3. The method of claim 1 wherein the urine is collected using a catheter inserted into the animal's bladder.

4. The method of claim 1 wherein the urine is collected using supra pubic aspiration

5. The method of claim 1 wherein the animal's metabolism is determined to be ketogenic if the amount of beta-hydroxy butyrate in the second urine sample exceeds the amount of beta-hydroxy butyrate in the first urine sample by at least one of 10%, 25%, 50%, 75%, 100%, 200%, 300%, 400%, and 500%.

6. A method for evaluating the effect of a comestible composition on the ketogenic status of an animal comprising:
collecting a first urine sample from the animal before feeding the comestible composition to the animal;
determining the concentration of beta-hydroxy butyrate in the first urine sample;
feeding the comestible composition to the animal;
collecting a second urine sample from the animal after feeding the comestible composition to the animal;
determining the concentration of beta-hydroxy butyrate in the second urine sample; and
concluding that the comestible composition caused the animal's metabolism to become ketogenic if the concentration of beta-hydroxy butyrate in the second urine sample exceeds the concentration of beta-hydroxy butyrate in the first urine sample by ten percent (10%) or more.

7. The method of claim 6 wherein the urine is collected by having the animal urinate into a container.

8. The method of claim 6 wherein the urine is collected using a catheter inserted into the animal's bladder.

9. The method of claim 6 wherein the urine is collected using supra pubic aspiration

10. The method of claim 6 wherein the comestible composition caused the animal's metabolism to become ketogenic if the concentration of beta-hydroxy butyrate in the second urine sample exceeds the concentration of beta-hydroxy butyrate in the first urine sample by at least one of 10%, 25%, 50%, 75%, 100%, 200%, 300%, 400%, and 500% .

11. A method for evaluating the effect of a diet on the ketogenic status of an animal comprising:
collecting a first urine sample from the animal before feeding the diet to the animal;
determining the concentration of beta-hydroxy butyrate in the first urine sample;
feeding the diet to the animal;
collecting a second urine sample from the animal while feeding the diet to the animal or after feeding the diet to the animal;
determining the concentration of beta-hydroxy butyrate in the second urine sample; and
concluding that the diet caused the animal's metabolism to become ketogenic if the concentration of beta-hydroxy butyrate in the second urine sample exceeds the concentration of beta-hydroxy butyrate in the first urine sample by ten percent (10%) or more.

12. The method of claim 9 wherein the urine is collected by having the animal urinate into a container.

13. The method of claim 9 wherein the urine is collected using a catheter inserted into the animal's bladder.

14. The method of claim 9 wherein the urine is collected using supra pubic aspiration

15. The method of claim 9 wherein the diet caused the animal's metabolism to become ketogenic if the concentration of beta-hydroxy butyrate in the second urine sample exceeds the concentration of beta-hydroxy butyrate in the first urine sample by at least one of 10%, 25%, 50%, 75%, 100%, 200%, 300%, 400%, and 500%.
